(19) United States
(12) Patent Application Publication
(10) Pub. No.: US 2022/0015631 A1
(43) Pub. Date: Jan. 20, 2022

(54) SINGLE-USE CONTACT TIP FOR TONOMETER

(71) Applicant: Reichert, Inc., Depew, NY (US)

(72) Inventors: David A. TAYLOR, Boston, NY (US); Mary M. MURPHY, Cheektowaga, NY (US)

(21) Appl. No.: 16/932,265

(22) Filed: Jul. 17, 2020

(51) Int. Cl. A61B 3/16; A61L 31/14; A61L 24/02; A61L 31/06; C09D 105/02; C09D 7/63

(52) U.S. Cl. CPC A61B 3/16; A61L 31/14; A61L 24/02; C08K 5/1545; C09D 105/02; C09D 7/63; A61L 31/06

(57) ABSTRACT

A contact tip for a contact tonometer has a body including a contact surface, and a coating applied to the contact surface. The coating includes a light-activated material and a biocompatible water soluble adhesive for adhering the coating to the contact surface. The water soluble adhesive dissolves upon contact with the corneal tear film during a tonometric measurement, thereby releasing the light-activated material into the tear film. The present disclosure further provides a single-use tonometer contact tip product which includes a sterilized contact tip having the mentioned contact surface coating, and an opaque package containing the sterilized contact tip.

### FIELD OF THE DISCLOSURE

The present disclosure relates to contact tonometers for measuring intraocular pressure (IOP).

### BACKGROUND OF THE DISCLOSURE

Contact tonometers, such as the Goldmann tonometer, have a contact tip for contacting the eye of a test subject to temporarily deform the cornea during an IOP measurement. The contact tip may have a body formed of a material which transmits light, wherein the body includes a contact surface which is pressed against the tear film on the cornea to deform the cornea. By use of a slit lamp, the operator views the tip-cornea contact region while varying the applied force on the contact tip to deform the cornea. When the deformed corneal area coincides with a standard calibration image of the tip, the applied force is measured and recorded for correlation to IOP. Viewing of the corneal contact region is enhanced by applying a light-activated material such as fluorescein dye to the tear film on the cornea.

Preventing infection is a primary safety consideration associated with a contact tonometry procedure. It is known to sterilize the contact tip before a tonometry procedure. It is also known to provide sterilized single-use contact tips made from inexpensive polymers, whereby the contact tip may be discarded after it is used on a test subject.

In addition to the contact tip, the applied light-activated material, e.g. fluorescein solution, should be provided in a sterile manner. It is known to apply fluorescein solution using a dropper bottle or a paper staining strip. This step takes time, effort and skill by the operator. Moreover, application by dropper bottle or staining strip is imprecise with regard to the amount of light-activated material delivered, and thus increases undesirable variability in the measurement results.

Attempts have been made to provide light-activated material to enhance visualization by incorporating the light-activated material into structure associated with the contact tip of the tonometer. U.S. Pat. No. 5,318,029 to Palese describes a disposable tip cover made of a flexible material that fits over a tonometer contact tip, wherein a light-activated material such as fluorescein is incorporated into the flexible material used to form the tip cover. Palese teaches that the light-activated material may be blended with the material used to form the tip cover, or may be applied to an inner surface of the tip cover between the tip cover and the contact tip of the tonometer, such that the light-activated material remains with the tip cover and is not dispersed into the tear film of the eye. Similar to Palese, U.S. Pat. No. 5,343,861 to Herman teaches a disposable tonometer tip cover in which light-activated material is added to uncured silicone material used to form the tip cover, such that the light-activated material is incorporated into the tip cover upon curing. Here again, the light-activated material remains with the tip cover and is not dispersed into the tear film of the eye. As a result, the disclosures mentioned above exhibit diminished visualization as compared to delivery of light-activated material into the tear film, and have not been widely adopted.

U.S. Pat. No. 5,540,227 to Poole describes a method whereby a solution containing a light-activated material and a disinfectant is applied by the operator to the contact surface of a reusable contact tip using a special applicator in preparation for a tonometric measurement procedure. According to the method, the operator must wait for the disinfectant to evaporate and leave a thin film of residual light-activated material on the contact surface of the contact tip before taking the measurement. During the measurement, the light-activated material on the contact surface is dissolved into the tear film. Perhaps owing to the delay between applying the solution to the contact surface and taking the measurement, and/or a tendency for a portion of the dried residue to blow or fall off the contact surface as the contact tip is positioned for measurement, the disclosed method has not gained widespread acceptance.

### SUMMARY OF THE DISCLOSURE

The present disclosure provides a contact tip for a contact tonometer. The contact tip generally comprises a body including a contact surface, and a coating applied to the contact surface. The coating includes a light-activated material and a biocompatible water soluble adhesive for adhering the coating to the contact surface. The water soluble adhesive dissolves upon contact with the corneal tear film during a tonometric measurement, thereby releasing the light-activated material into the tear film.

In one embodiment, the adhesive may comprise sodium chloride. In another embodiment, the adhesive may comprise dextran. The light-activated material may be, for example, fluorescein.

The contact surface may be planar as in traditional Goldmann tonometry. Alternatively, the contact surface may be concave or may be convex.

The present disclosure further provides a single-use tonometer contact tip product that comprises a tonometer contact tip as summarized above which is sterilized, and an opaque package containing the sterilized tonometer contact tip.

### BRIEF DESCRIPTION OF THE DRAWING VIEWS

The nature and mode of operation of the present invention will now be more fully described in the following detailed description taken with the accompanying drawing figures, in which:

FIG. 1 is a perspective view showing a contact tip for a contact tonometer formed in accordance with an embodiment of the present disclosure;

FIG. 2 is an enlarged side elevation view showing a distal portion of the contact tip of FIG. 1 in greater detail;

FIG. 3 is a view similar to that of FIG. 2, but showing a variation in a contact surface of the contact tip in accordance with an alternative embodiment;

FIG. 4 is another view similar to that of FIG. 2, but showing a further variation in a contact surface of the contact tip in accordance with another alternative embodiment; and

FIG. 5 is a perspective view showing a single-use tonometer contact tip product in accordance with a further embodiment of the present disclosure.

### DETAILED DESCRIPTION

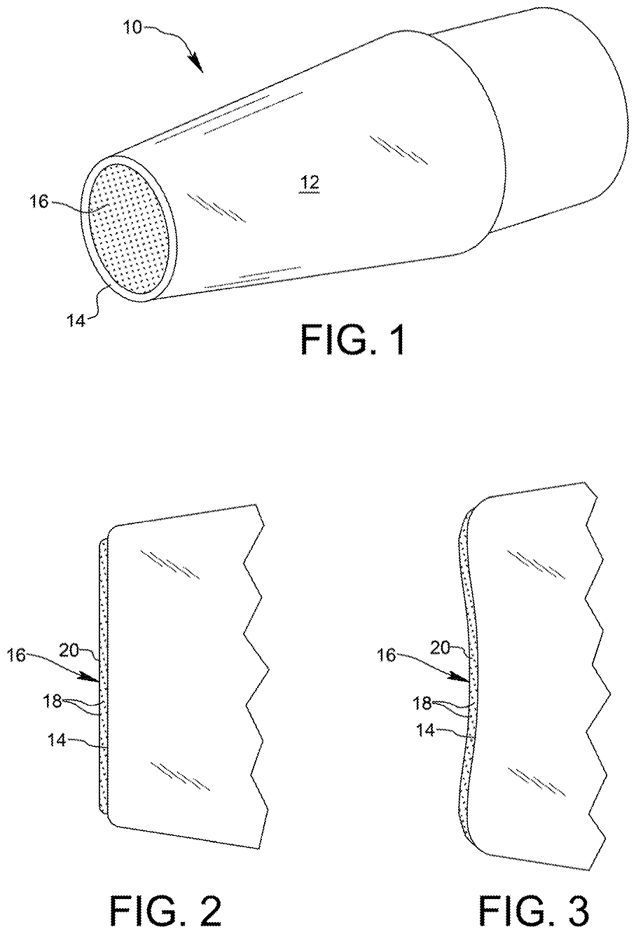

FIG. 1 shows a tonometer contact tip 10 formed in accordance with an embodiment of the present disclosure. Contact tip 10 is configured for removable mounting on a contact tonometer for use in taking a contact tonometer measurement of TOP of a test subject. For example, contact tip may be configured to mount on a CT100 Contact Tonometer or a CT210 Contact Applanation Tonometer manufactured by the current applicant, Reichert, Inc. Contact tip 10 may be configured to mount on other contact tonometers available from other manufacturers.

Contact tip 10 comprises a body 12 including a contact surface 14 at a distal portion of the contact tip. Body 12 may be formed of a material which transmits light. For example, body 12 may be formed of an inexpensive light-transmitting polymer such as Polymethyl methacrylate (PMMA).

As may be seen in FIG. 2, contact tip 10 further comprises a coating 16 applied to contact surface 14, wherein the coating includes a light-activated material 18 and a biocompatible water soluble adhesive 20 for adhering the coating to the contact surface. As may be understood, the water soluble adhesive 20 dissolves upon contact with the corneal tear film of a test subject during a tonometric measurement, thereby releasing the light-activated material 18 into the tear film.

In one embodiment, the water soluble adhesive 20 may comprise sodium chloride. The light-activated material 18 may be fluorescein. For example, coating 16 may be applied to contact surface 14 as a solution of 0.5 M sodium chloride and 0.5 mg/mL fluorescein in water, and the water may be allowed to evaporate from coating 16 leaving a thin dry film of sodium chloride and fluorescein on contact surface 14.

In another embodiment the water soluble adhesive 20 may comprise dextran. The light-activated material 18 may be fluorescein. For example, coating 16 may be applied to contact surface 14 by spraying a solution of dextran in water onto contact surface 14, adding a predetermined amount of fluorescein flakes or powder to the applied solution on the contact surface, and allowing the water in the applied solution to evaporate from coating 16 leaving a thin dry film of dextran and fluorescein on contact surface 14. For example, the applied solution may be 20% weight/volume of dextran in water, and the predetermined amount of fluorescein may be 0.5 mg or approximately 0.5 mg (i.e. 0.5 mg+/−0.1 mg).

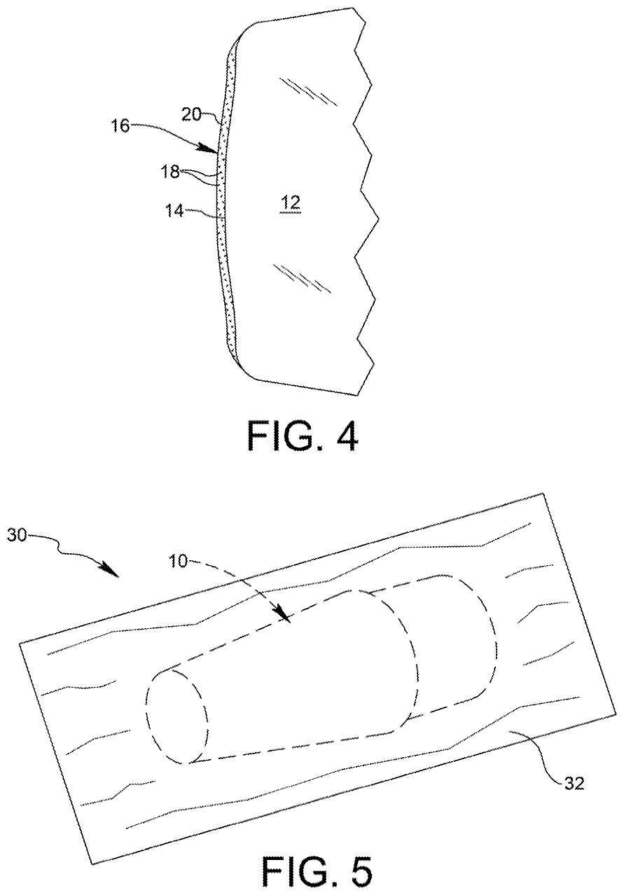

As shown in FIG. 2, contact surface 14 may be planar. Alternatively, contact surface 14 may be non-planar. For example, as shown in FIG. 3, contact surface 14 may be concave, and as shown in FIG. 4, contact surface 14 may be convex.

Referring now to FIG. 5, a single-use tonometer contact tip product in accordance with a further embodiment of the present disclosure is shown and generally identified by reference numeral 30. Single-use product 30 comprises a tonometer contact tip 10 as described above, wherein the contact tip 10 is sterilized, and further comprises an opaque package 32 containing the sterilized tonometer contact tip 10. Due to the opaque package 32, contact tip 10 remains sterile and the light-activated material 18 adhered to contact surface 14 is not exposed to light while the sterilized tonometer contact tip 10 is in the opaque package. Opaque package 32 may be, for example, a thermoformed TYVEK® lidded tray.

The present disclosure provides a single-use contact tip for contact tonometry that eliminates the imprecise and time-consuming step of applying a solution containing light-activated material to an eye of a test subject using a dropper bottle or a paper staining strip, or to a contact surface of a reusable contact tip using an applicator, and reliably delivers a consistent amount of light-activated material to an eye of a test subject. As a result, measurement efficiency and repeatability are improved.

While the present disclosure describes exemplary embodiments, the detailed description is not intended to limit the scope of the appended claims to the particular embodiments set forth. The claims are intended to cover such alternatives, modifications and equivalents of the described embodiments as may be included within the scope of the claims.

What is claimed is:

1. A contact tip for a contact tonometer, the contact tip comprising:
a body including a contact surface;
a coating applied to the contact surface, the coating including a light-activated material and a biocompatible water soluble adhesive for adhering the coating to the contact surface;
wherein the water soluble adhesive dissolves upon contact with the corneal tear film during a tonometric measurement, thereby releasing the light-activated material into the tear film.

2. The contact tip according to claim 1, wherein the adhesive comprises sodium chloride.

3. The contact tip according to claim 2, wherein the light-activated material is fluorescein.

4. The contact tip according to claim 3, wherein the coating is applied to the contact surface as a solution of sodium chloride and fluorescein in water, and the water evaporates from the coating.

5. The contact tip according to claim 4, wherein the applied solution is 0.5 M sodium chloride and 0.5 mg/mL fluorescein in water.

6. The contact tip according to claim 1, wherein the adhesive comprises dextran.

7. The contact tip according to claim 6, wherein the light-activated material is fluorescein.

8. The contact tip according to claim 6, wherein the coating is applied to the contact surface by applying a solution of dextran in water to the contact surface and adding a predetermined amount of fluorescein to the applied solution, and the water evaporates from the coating.

9. The contact tip according to claim 8, wherein the applied solution is 20% weight/volume of dextran in water.

10. The contact tip according to claim 9, wherein the predetermined amount of fluorescein is approximately 0.5 mg.

11. The contact tip according to claim 1, wherein the contact surface is planar.

12. The contact tip according to claim 1, wherein the contact surface is concave.

13. The contact tip according to claim 1, wherein the contact surface is convex.

14. The contact tip according to claim 1, wherein the body is formed of a material which transmits light.

15. A single-use product comprising:
a tonometer contact tip according to claim 1, wherein the contact tip is sterilized; and
an opaque package containing the sterilized tonometer contact tip, wherein the light-activated material is not exposed to light while the sterilized tonometer contact tip is in the opaque package.
